(19) United States
(12) Patent Application Publication
(10) Pub. No.: US 2014/0296681 A1
(43) Pub. Date: Oct. 2, 2014

(54) ELECTROCARDIOGRAPH, METHOD FOR MEASURING ELECTROCARDIOGRAM, AND COMPUTER PROGRAM PRODUCT

(71) Applicant: KABUSHIKI KAISHA TOSHIBA, Tokyo (JP)

(72) Inventors: Kanako Nakayama, Shinagawa-ku (JP); Takuji Suzuki, Yokohama-shi (JP); Sawa Fuke, Kawasaki-shi (JP)

(73) Assignee: KABUSHIKI KAISHA TOSHIBA, Tokyo (JP)

(21) Appl. No.: 14/196,008

(22) Filed: Mar. 4, 2014

(30) Foreign Application Priority Data
Apr. 1, 2013 (JP) 2013-075631

(51) Int. Cl. A61B 5/0408

(52) U.S. Cl. CPC A61B 5/04085
USPC 600/382

(57) ABSTRACT

According to an embodiment, an electrocardiograph includes first and second electrode pairs, first and second detectors, and an electrocardiogram detector. A difference between a first distance between two electrodes of the first electrode pair on a first line and a second distance between two electrodes of the second electrode pair on a second line is not more than a first threshold. An angle formed by the first and second lines is not less than a second threshold. The first detector is configured to detect a first differential electric potential of the first electrode pair. The second electric potential detector is configured to detect a second differential electric potential of the second electrode pair. The electrocardiogram detector is configured to detect an electrocardiogram by performing a subtraction process on the first and second differential electric potentials.

### CROSS-REFERENCE TO RELATED APPLICATION

This application is based upon and claims the benefit of priority from Japanese Patent Application No. 2013-075631, filed on Apr. 1, 2013; the entire contents of which are incorporated herein by reference.

### FIELD

Embodiments described herein relate generally to an electrocardiograph, a method for measuring an electrocardiogram, and a computer program product.

### BACKGROUND

Recently, awareness of health care has been increasing. In accordance with this, an electrocardiograph that allows electrocardiographic measurement in daily life has been proposed. This electrocardiograph typically performs electrocardiographic measurement by disposing electrodes with sandwiching a heart and measuring a bioelectric potential.

However, the conventional electrocardiograph requires, for example, expertise and guidance by a doctor and fastening the electrodes using, for example, a belt during installation. Accordingly, an examinee is difficult to perform electrocardiographic measurement in daily life without burden.

### BRIEF DESCRIPTION OF THE DRAWINGS

FIG. 1 illustrates an exemplary hardware configuration of an electrocardiograph according to an embodiment;

FIGS. 2A and 2B illustrate exemplary appearances (1) of the electrocardiograph according to the embodiment;

FIG. 3 illustrates an exemplary installation of the electrocardiograph according to the embodiment;

FIG. 4 illustrates an exemplary appearance (2) of the electrocardiograph according to the embodiment;

FIG. 5 illustrates an exemplary appearance (3) of the electrocardiograph according to the embodiment;

FIG. 6 illustrates an exemplary functional configuration of electrocardiographic measurement according to the embodiment;

FIG. 7 is a schematic diagram of a muscle;

FIG. 8 illustrates an exemplary method for detecting the electrocardiogram according to the embodiment;

FIG. 9 illustrates exemplary waveforms of a bioelectric potential and an electrocardiogram according to the embodiment;

FIGS. 10A and 10B illustrate exemplary arrangements of each electrode according to the embodiment;

FIG. 11 illustrates a flowchart of an exemplary process procedure for detecting electrocardiogram according to the embodiment;

FIG. 12 illustrates an exemplary functional configuration of electrocardiographic measurement of Modification 1;

FIG. 13 illustrates an exemplary waveform of an electrocardiogram of Modification 1; and

FIG. 14 illustrates an exemplary functional configuration of electrocardiographic measurement of Modification 2.

### DETAILED DESCRIPTION

According to an embodiment, an electrocardiograph includes a first electrode pair, a second electrode pair, a first electric potential detector, a second electric potential detector, and an electrocardiogram detector. The first electrode pair includes a first measurement electrode and a first reference electrode. The first measurement electrode is apart from the first reference electrode with a first distance on a first line. The second electrode pair includes a second measurement electrode and a second reference electrode. The second measurement electrode is apart from the second reference electrode with a second distance on a second line. A difference between the first distance and the second distance is equal to or less than a first threshold. An angle formed by the first line related to the first electrode pair and the second line related to the second electrode pair is equal to or more than a second threshold. The first electric potential detector is configured to detect a first differential electric potential of the first electrode pair. The second electric potential detector is configured to detect a second differential electric potential of the second electrode pair. The electrocardiogram detector is configured to detect an electrocardiogram by performing a subtraction process on the first differential electric potential and the second differential electric potential.

The following describes embodiments of an electrocardiograph, a method for measuring electrocardiogram, and an electrocardiographic program in detail with reference to the accompanying drawings.

Electrocardiograph

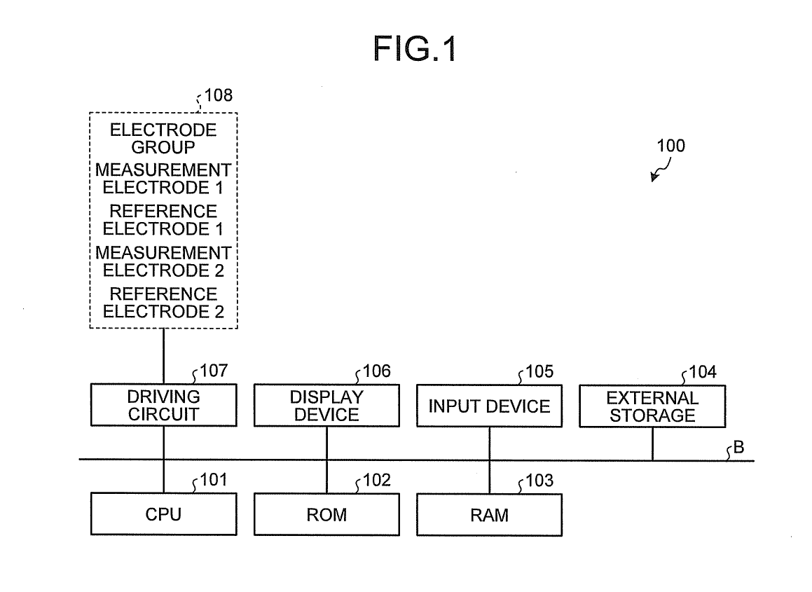

FIG. 1 illustrates an exemplary hardware configuration of an electrocardiograph 100 of an embodiment. As illustrated in FIG. 1, the electrocardiograph 100 of the embodiment includes, for example, a Central Processing Unit (CPU) 101, a Read Only Memory (ROM) 102, a Random Access Memory (RAM) 103, an external storage 104, an input device 105, and a display device 106. In the electrocardiograph 100 of the embodiment, each hardware is coupled via a bus B.

The CPU 101 is an arithmetic device that controls the entire device and achieves equipped functions. The ROM 102 is a non-volatile semiconductor memory storing, for example, a program achieving functions and function setting data. The RAM 103 is a volatile semiconductor memory from which a program and data are read and where the program and data are temporarily held. The CPU 101, for example, reads the program and data from the ROM 102 on the RAM 103 and achieves a control of the entire device and the equipped functions by performing processes.

The external storage 104, for example, is a non-volatile memory such as a Hard Disk Drive (HDD) and a memory card. The external storage 104 includes a storage medium such as a flexible disk (FD), a Compact Disk (CD), and a Digital Versatile Disk (DVD). The input device 105 is a numeric keypad and a touchscreen, for example, and is used for input of each operation signal to the electrocardiograph 100. The display device 106 is a display, for example, and displays a result of a process by the electrocardiograph 100.

The electrocardiograph 100 of the embodiment includes at least four electrodes 108 (hereinafter referred to as “electrode group 108”) including a measurement electrode 1, a reference electrode 1, a measurement electrode 2, and a reference electrode 2; and a driving circuit 107. In the electrocardiograph 100 of the embodiment, the driving circuit 107 is coupled via the bus B.

The electrode group 108 detects a bioelectric potential by contacting the skin of the examinee. The driving circuit 107 drives each electrode. The driving circuit 107 outputs the detected bioelectric potential value obtained from the electrode group 108 to, for example, the CPU 101 via the bus B.

Here, appearances and installation examples of the electrocardiograph 100 of the embodiment and exemplary arrangements of the electrode group 108 will be described.

Appearance and Exemplary Arrangement 1

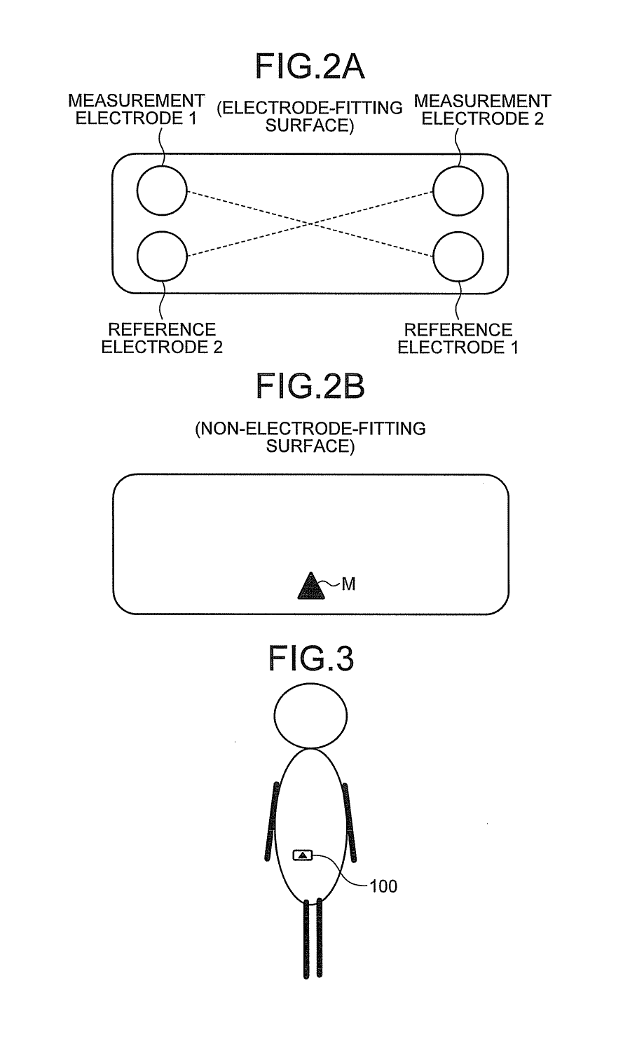

FIG. 2A and FIG. 2B illustrate exemplary appearances (1) of the electrocardiograph 100 according to the embodiment. FIG. 3 illustrates an exemplary installation of the electrocardiograph 100 according to the embodiment. As illustrated in FIG. 2A, in this embodiment, the electrocardiograph 100 includes the measurement electrode 1, the reference electrode 1, the measurement electrode 2, and the reference electrode 2 on the surface contacting the skin of the examinee during measurement. In the following description, the surface contacting the skin of the examinee during measurement is referred to as an electrode-fitting surface. The electrode-fitting surface employs a rectangular shape. As illustrated in FIG. 2B, in this embodiment, the electrocardiograph 100 includes a mark M showing a vertical direction during installation on the opposite surface of the electrode-fitting surface (hereinafter referred to as a non-electrode-fitting surface). The expression of the vertical direction is not limited to the mark M and may be expressed by a character, for example, showing the vertical direction.

Thus, the examinee installs the electrocardiograph 100 as illustrated in FIG. 3 as follows. The upper direction indicated by the mark M is set at the head side and the lower direction indicated by the mark M is set at the leg side. Then, the electrocardiograph 100 is attached to, a rubber and a belt of clothing, such as trousers such that the electrode group 108 on the electrode-fitting surface can contact around the navel at the abdomen. Thus, the electrocardiograph 100 of the embodiment with the rectangular electrode-fitting surface allows improving installability to the examinee. The electrocardiograph 100 of the embodiment includes the mark M, which indicates the vertical direction, on the non-electrode-fitting surface. This prevents incorrect installation by the examinee.

Exemplary Arrangements of Electrode Group 108

As illustrated in FIG. 2A, the electrocardiograph 100 of the embodiment includes the measurement electrode 1, the reference electrode 1, the measurement electrode 2, and the reference electrode 2 on the apexes of the rectangular electrode-fitting surface. The two sets of electrode pairs: the measurement electrode 1 and the reference electrode 1, and the measurement electrode 2 and the reference electrode 2, are cater-cornered on the rectangular electrode-fitting surface. FIG. 2A illustrates an exemplary arrangement where the measurement electrodes 1 and 2 are positioned in the upper direction and the reference electrodes 1 and 2 are positioned in the lower direction during installation. However, this should not be construed in a limiting sense. The reference electrodes 1 and 2 may be positioned in the upper direction and the measurement electrodes 1 and 2 may be positioned in the lower direction (arrangements of the measurement electrodes 1 and 2 and the reference electrodes 1 and 2 may be upside down) during installation, for example.

Appearances and Exemplary Arrangements 2 and 3

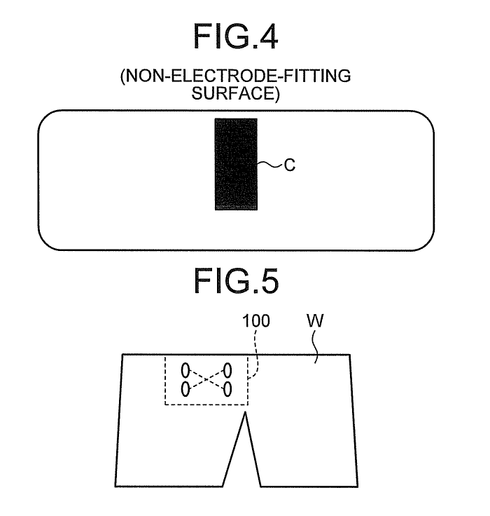

FIG. 4 and FIG. 5 illustrate exemplary appearances (2 and 3) of the electrocardiograph 100 of the embodiment. The electrocardiograph 100 of the embodiment, for example, may include a clip C or similar member on the non-electrode-fitting surface as illustrated in FIG. 4. With the electrocardiograph 100, the examinee sandwiches the rubber and the belt of the clothing, such as trousers, with the clip C such that the electrode group 108 on the electrode-fitting surface may contact around the navel at the abdomen, thus the electrocardiograph 100 is installed. Thus, the electrocardiograph 100 of the embodiment includes an attachment for securing the electrocardiograph 100. This prevents the electrode group 108 on the electrode-fitting surface from detaching and dropping from the living body due to the body motion of the examinee.

The electrocardiograph 100 of the embodiment may be built into clothing W itself as illustrated in FIG. 5. In this case, the electrode group 108 is preferred to be attachable/removable from the electrocardiograph 100.

Some conventional measuring instruments are installed to a chest for electrocardiographic measurement. However, this measuring instrument requires, for example, a belt to secure the electrodes for installation, complicated work for the examinee. Some of the conventional measuring instruments are installed to an arm for electrocardiographic measurement. However, this measuring instrument is displaced due to the body motion of the examinee and may fail the electrocardiographic measurement.

In contrast to this, the electrocardiograph 100 of the embodiment allows simple installation with the configuration, providing an electrocardiographic measurement environment available for daily use for the examinee.

Electrocardiographic Measurement Function

The electrocardiographic measurement function of the embodiment will be described. The electrocardiograph 100 of the embodiment includes at least two sets of electrode pairs, the measurement electrode 1 and the reference electrode 1; and the measurement electrode 2 and the reference electrode 2. Each electrode pair of the measurement electrode 1 and the reference electrode 1 and the measurement electrode 2 and the reference electrode 2 of the embodiment are arranged on the electrode-fitting surface considering the direction of the muscle fiber and the transmission direction of electrocardiogram at the installation position on the body member of the examinee (installation position of the electrocardiograph 100 during measurement). The electrocardiograph 100 of the embodiment detects differential electric potentials between electrodes of the measurement electrode 1 and the reference electrode 1 and between electrodes of the measurement electrode 2 and the reference electrode 2 as two sets of bioelectric potentials, respectively. The electrocardiograph 100 of the embodiment performs a subtraction process on the two sets of bioelectric potentials and detects an electrocardiogram. The electrocardiograph 100 of the embodiment features such electrocardiographic measurement function.

The conventional measuring instrument may cause reduction in measurement accuracy by generating noise of, for example, myoelectricity by motion of the muscle fiber at the installation position and due to smallness of detected bioelectric potentials (amplitude of electrocardiographic complex is small) compared with the electrocardiographic measurement sandwiching the heart with electrodes.

Therefore, the electrocardiograph of the embodiment detects each differential electric potential of the two sets of electrode pairs arranged on the electrode-fitting surface considering the direction of the muscle fiber and the transmission direction of electrocardiogram at the installation position as bioelectric potentials. The electrocardiograph of the embodiment performs a subtraction process on the detected two sets of bioelectric potentials and detects an electrocardiogram.

The following describes the configuration and the operation of the electrocardiographic measurement function of the embodiment.

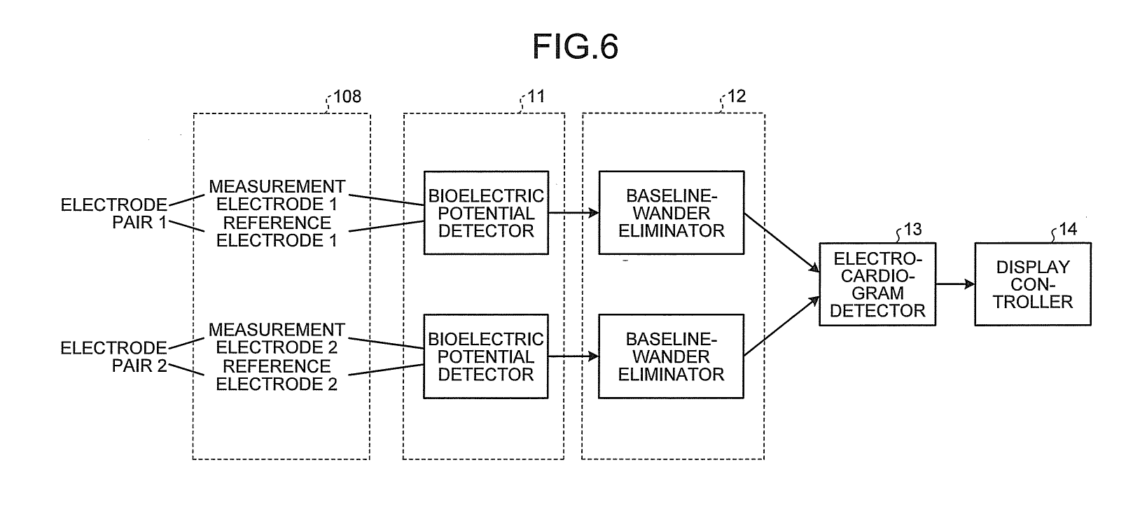

FIG. 6 illustrates an exemplary functional configuration of electrocardiographic measurement according to the embodiment. As illustrated in FIG. 6, the electrocardiographic measurement function of the embodiment includes, for example, a bioelectric potential detector 11, a baseline-wander eliminator 12, an electrocardiogram detector 13, and a display controller 14. The bioelectric potential detector 11 is a functional unit that detects each differential electric potential between electrodes of the measurement electrode 1 and the reference electrode 1 and between electrodes of the measurement electrode 2 and the reference electrode 2 as two sets of bioelectric potentials. The baseline-wander eliminator 12 is a functional unit that eliminates high-frequency components of the detected twos sets of bioelectric potentials and baseline wander. The electrocardiogram detector 13 is a functional unit that performs a subtraction process on an output after the baseline wander is eliminated and detects an electrocardiogram. The display controller 14 is a functional unit that controls the display device 106 to display a measurement result of, for example, detected electrocardiographic complex.

The bioelectric potential detector 11 detects the two sets of differential electric potentials of each electrode pair 1 and 2 based on an electric potential measured at the electrode pair 1 of the measurement electrode 1 and the reference electrode 1 (first electrode pair) and the electrode pair 2 of the measurement electrode 2 and the reference electrode 2 (second electrode pair). Then, the bioelectric potential detector 11 obtains a difference between an electric potential measured at the measurement electrode 1 and an electric potential measured at the reference electrode 1 and detects a differential electric potential between the electrodes of the measurement electrode 1 and the reference electrode 1 (first electric potential). The bioelectric potential detector 11 obtains a difference between an electric potential measured at the measurement electrode 2 and an electric potential measured at the reference electrode 2 and detects a differential electric potential between the electrodes of the measurement electrode 2 and the reference electrode 2 (second electric potential). Thus, the bioelectric potential detector 11 detects each detected differential electric potential as two sets of bioelectric potentials.

The baseline-wander eliminator 12, for example, eliminates extra high-frequency components from the waveform of the detected bioelectric potentials by a low-pass filter function at a cutoff frequency of 15 [Hz]. Then, the baseline-wander eliminator 12 eliminates baseline wander by performing a first derivation process on the waveforms after the high-frequency components are eliminated.

The electrocardiogram detector 13 performs a subtraction process on an output after baseline wander is eliminated (two sets of bioelectric potentials) and detects an electrocardiogram with myoelectricity eliminated.

The following describes a method for detecting an electrocardiogram of the embodiment.

Method for Detecting Electrocardiogram

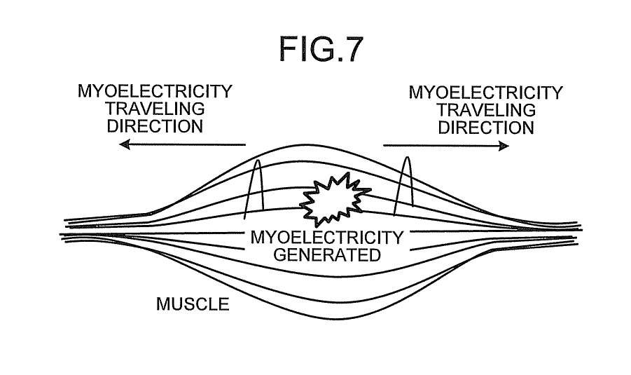

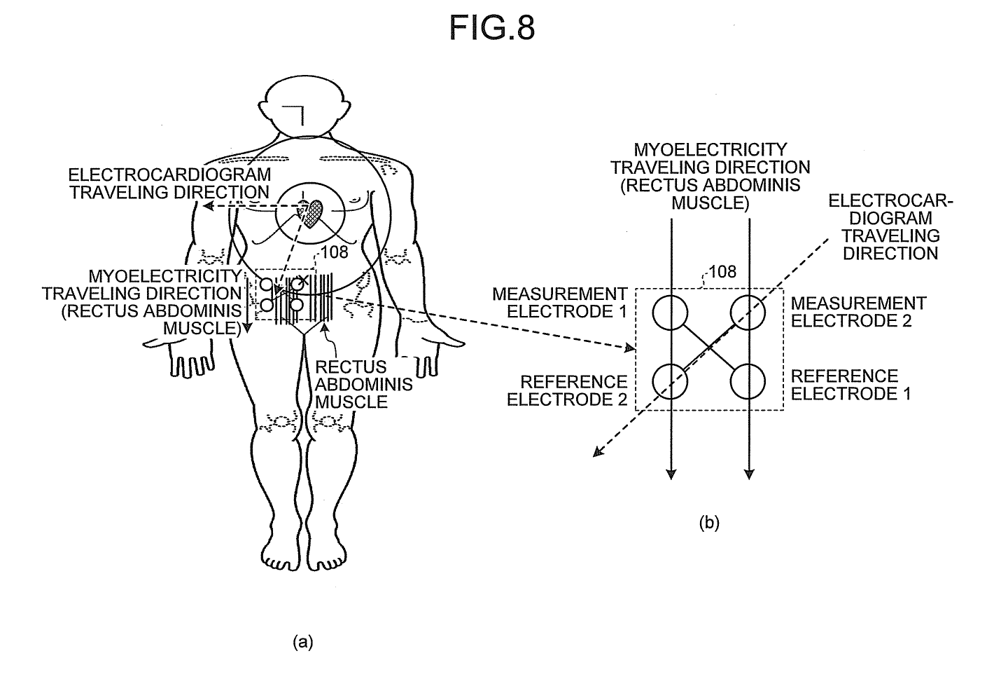

FIG. 7 is a schematic diagram of a muscle. FIG. 8 illustrates an exemplary method for detecting the electrocardiogram of the embodiment. As illustrated in FIG. 7, myoelectricity is generated together with the motion of the muscle and is transmitted to the myoelectricity traveling direction, which is a direction from near the center of the muscle toward the muscle fiber. As illustrated in (a) of FIG. 8, the human abdomen includes a muscle referred to as rectus abdominis muscle, which supports the body, in the longitudinal direction.

Accordingly, the myoelectricity of rectus abdominis muscle transmits to the direction of the fiber of rectus abdominis muscle, that is, a linear direction connecting from the head to the leg of the living body (direction indicated by solid line arrows in the drawing). Therefore, in this embodiment, as illustrated in (b) of FIG. 8, myoelectricity with approximately same electric potential is detected from the electrode pair 1 of the measurement electrode 1 and the reference electrode 1 and the electrode pair 2 of the measurement electrode 2 and the reference electrode 2.

As illustrated in (a) of FIG. 8, the electrocardiogram is an electric potential generated by the muscle of the heart and the source of generation is the heart. The electrocardiogram concentrically transmits on the surface of the living body centering the heart (direction indicated by the dotted line arrow in the drawing). Accordingly, as illustrated in (a) of FIG. 8, in the case where the electrode group 108 is disposed side to the navel, for example, the electrocardiogram transmits in the direction as follows. The electrocardiogram, as illustrated in (b) of FIG. 8, transmits to the direction connecting the measurement electrode 2 and the reference electrode 2 (direction indicated by the dotted line arrow in the drawing). This is because the electrode pair 2 of the measurement electrode 2 and the reference electrode 2 is disposed so as to be approximately parallel to the transmission direction of the electrocardiogram. Therefore, in this embodiment, the transmitted electrocardiogram varies the electric potentials between the electrodes of the measurement electrode 2 and the reference electrode 2, thus electrocardiogram with large amplitude is detected from the differential electric potential between the electrodes. On the other hand, since the direction connecting the measurement electrode 1 and the reference electrode 1 is positioned on the concentric circle centering the heart, the electric potential measured at the measurement electrode 1 and the electric potential measured at the reference electrode 1 are approximately same electric potential. This is because that the electrode pair 1 of the measurement electrode 1 and the reference electrode 1 is disposed so as to be approximately vertical to the transmission direction of the electrocardiogram. Accordingly, in this embodiment, the electrocardiogram is not detected from the differential electric potential between the electrodes of the measurement electrode 1 and the reference electrode 1.

Thus, in the electrocardiograph 100 of the embodiment, the electrode pair 2 of the measurement electrode 2 and the reference electrode 2 is disposed parallel (horizontal direction) to the transmission direction of the electrocardiogram when appropriately installed. The electrode pair 1 of the measurement electrode 1 and the reference electrode 1 is disposed vertical (vertical direction) to the transmission direction of the electrocardiogram.

Accordingly, the electrocardiograph 100 of the embodiment includes the same level of myoelectricity (same electric potential) in the differential electric potential between the electrodes of the measurement electrode 1 and the reference electrode 1 and the differential electric potential between the electrodes of the measurement electrode 2 and the reference electrode 2. In this embodiment, electrocardiogram is not included in the differential electric potential between the electrodes of the measurement electrode 1 and the reference electrode 1 but included in the differential electric potential between the electrodes of the measurement electrode 2 and the reference electrode 2.

The electrocardiogram detector 13 of the embodiment focuses on a difference in property of the differential electric potentials. The electrocardiogram from which myoelectricity is eliminated is detected by subtracting the differential electric potential not including electrocardiogram from the differential electric potential including electrocardiogram.

Electrocardiographic Complex

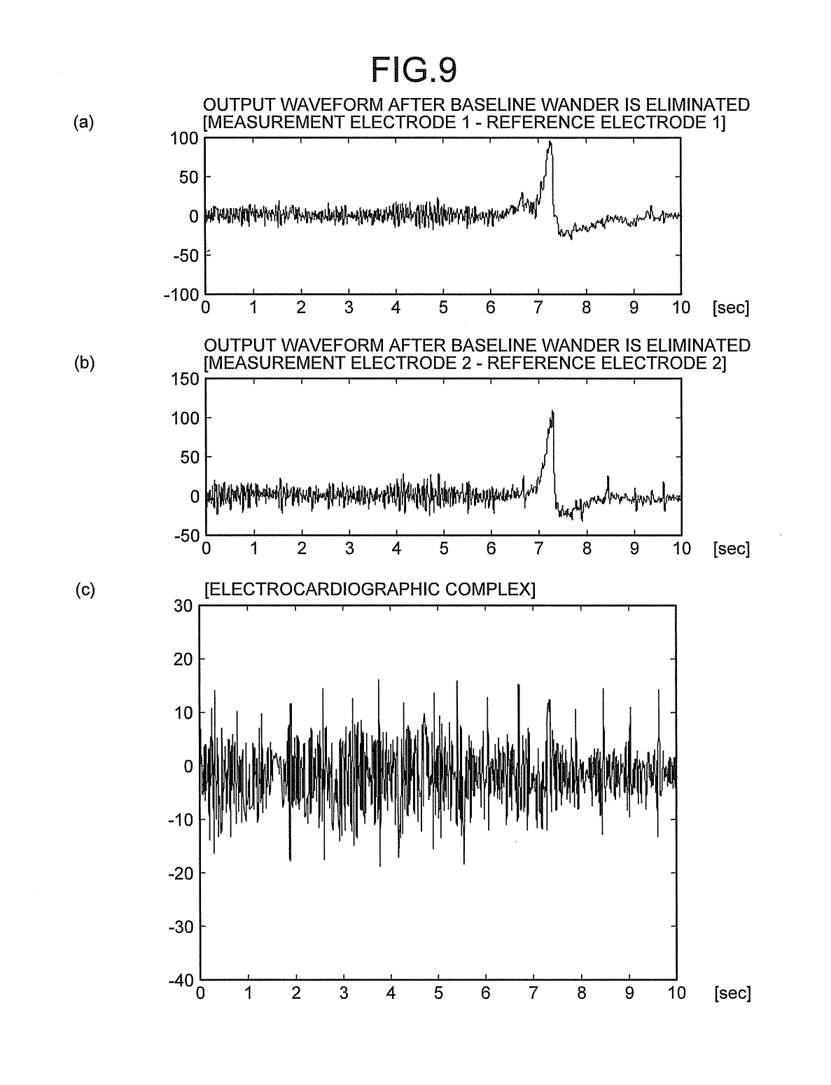

FIG. 9 illustrates exemplary waveforms of a bioelectric potential and an electrocardiogram of the embodiment. Illustrated in (a) of FIG. 9 is an output waveform (bioelectric potential waveform) after baseline wander removal process is performed on the differential electric potential (bioelectric potential) between the electrodes of the measurement electrode 1 and the reference electrode 1. Illustrated in (b) of FIG. 9 is an output waveform after baseline wander removal process is performed on the differential electric potential between the electrodes of the measurement electrode 2 and the reference electrode 2. Illustrated in (c) of FIG. 9 is electrocardiographic complex where myoelectricity is eliminated by performing a subtraction process on an output after baseline wander is eliminated.

Thus, after the removal process by the baseline-wander eliminator 12, the electrocardiographic measurement function of the embodiment outputs two sets of bioelectric potentials after baseline wander is eliminated, which are as illustrated in (a) and (b) of FIG. 9, from the baseline-wander eliminator 12 to the electrocardiogram detector 13. Then, the electrocardiogram detector 13 subtracts the bioelectric potential not including electrocardiogram from the bioelectric potential including electrocardiogram among the two sets of bioelectric potentials to detect an electrocardiogram (myoelectricity from which electrocardiogram is eliminated) as illustrated in (c) of FIG. 9. Afterwards, the detection result of the electrocardiogram is output from the electrocardiogram detector 13 to the display controller 14. Consequently, the display controller 14 displays the detection result of the electrocardiogram on the display device 106 as the measurement result of electrocardiogram.

Here, the arrangement of the electrode group 108 of the embodiment is additionally described. FIG. 2A illustrates an exemplary arrangement where the line segment connecting the measurement electrode 1 and the reference electrode 1 intersects with the line segment connecting the measurement electrode 2 and the reference electrode 2 at the center point of each line segment. However, this should not be construed in a limiting sense.

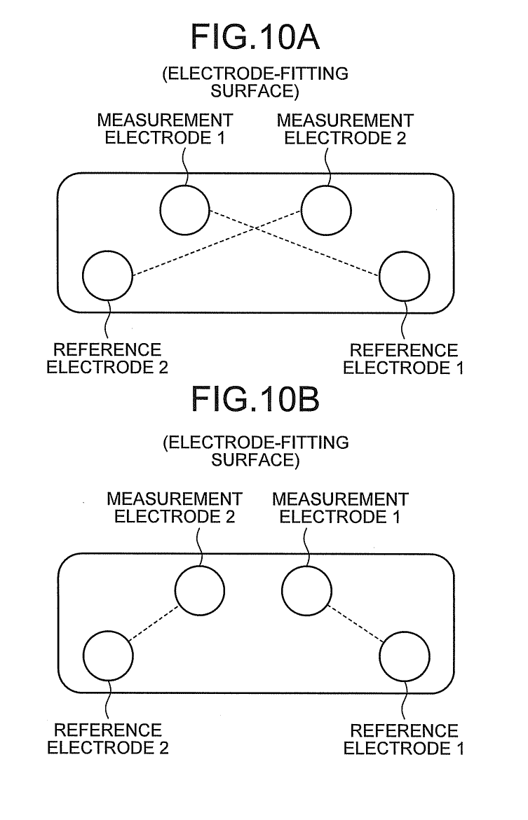

FIGS. 10A and 10B illustrate exemplary arrangements of each electrode of the embodiment. As illustrated in FIG. 10A, for example, the line segment connecting the measurement electrode 1 and the reference electrode 1 may not intersect with the line segment connecting the measurement electrode 2 and the reference electrode 2 at the center point of each line segment. As illustrated in FIG. 10B, the line segment connecting the measurement electrode 1 and the reference electrode 1 may not intersect with the line segment connecting the measurement electrode 2 and the reference electrode 2. Thus, the electrode group 108 of the embodiment has an arrangement where the line segment connecting the electrode pair 1 of the measurement electrode 1 and the reference electrode 1 forms an angle equal to or more than threshold with respect to the line segment connecting electrode pair 2 of the measurement electrode 2 and the reference electrode 2. It is only necessary that the electrode group 108 of the embodiment have an arrangement where one of the electrode pair 1 of the measurement electrode 1 and the reference electrode 1 and the electrode pair 2 of the measurement electrode 2 and the reference electrode 2 is disposed approximately parallel to the transmission direction of electrocardiogram while the other side is disposed approximately vertical to the transmission direction of the electrocardiogram.

However, as described in the method for detecting an electrocardiogram, regarding a distance between each electrode in the electrode group 108, to eliminate myoelectricity, between the electrodes of the measurement electrode 1 and the reference electrode 1, and between the electrodes of the measurement electrode 2 and the reference electrode 2, the distance where the same level of myoelectricity can be detected is required to be maintained. That is, each electrode is preferred to be disposed on the electrode-fitting surface to provide a distance contacting on the same rectus abdominis muscle in the case where the electrocardiograph 100 is appropriately installed. Therefore, the distance between each electrode in the electrode group 108 is preferred to be the same as or smaller than the length of the cross-sectional width of the rectus abdominis muscle. A distance between each electrode in the electrode group 108 is, for example, equal to or less than 50 [mm]. Thus, in the arrangement of the electrode group 108 of the embodiment, a first distance is kept between the electrodes of the measurement electrode 1 and the reference electrode 1, while a second distance is kept between the electrodes of the measurement electrode 2 and the reference electrode 2. The second distance is a distance where difference from the first distance is equal to or less than the threshold.

The electrocardiographic measurement function of the above-described embodiment can be achieved by executing an electrocardiograph program in the electrocardiograph 100, where the functional units each operate collaboratively.

The electrocardiographic program is provided by being preliminarily incorporated in the ROM 102 included in the electrocardiograph 100, which is execution environment. The electrocardiographic program has a module configuration including each of the functional units. The CPU 101 reads and executes the program from the ROM 102, thus generating each functional unit on the RAM 103. A method for providing the electrocardiographic program is not limited to this. The electrocardiographic program may be stored in a device coupled to, for example, the Internet, may be downloaded via network so as to be distributed, for example. An installable format or executable format file may be stored in a storage medium readable by the electrocardiograph 100 and may be provided as a computer program product.

The following describes a process during execution of the electrocardiographic program (collaborative operation by each functional unit) using a flowchart.

Process During Electrocardiographic Measurement

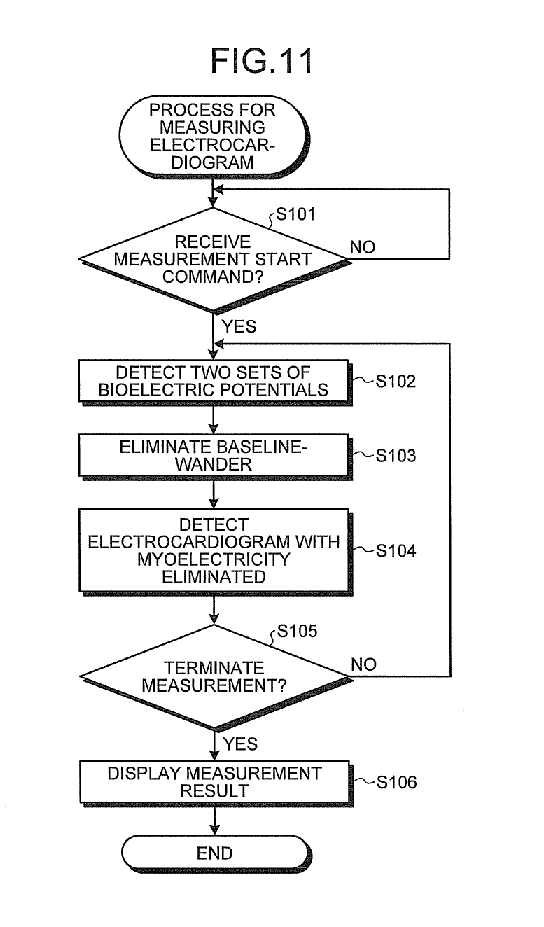

FIG. 11 illustrates a flowchart of an exemplary process procedure for detecting electrocardiogram of the embodiment. As illustrated in FIG. 11, the electrocardiograph 100 of the embodiment receives an electrocardiographic measurement start command from the examinee via the input device 105 (Step S101: YES). The electrocardiograph 100 of the embodiment stands by for electrocardiographic measurement to start while not receiving the electrocardiographic measurement start command (Step S101: NO).

Next, the bioelectric potential detector 11 detects bioelectric potentials from two sets of the electrode pairs 1 and 2 of the electrode group 108 (Step S102). Then, the bioelectric potential detector 11 obtains a difference in the electric potential measured at measurement electrode 1 and the electric potential measured at the reference electrode 1 and detects a differential electric potential between the electrodes of the measurement electrode 1 and the reference electrode 1. The bioelectric potential detector 11 obtains a difference in the electric potential measured at measurement electrode 2 and the electric potential measured at the reference electrode 2 and detects a differential electric potential between the electrodes of the measurement electrode 2 and the reference electrode 2. Accordingly, the bioelectric potential detector 11 detects each detected differential electric potential as two sets of bioelectric potentials.

Next, the baseline-wander eliminator 12 performs a baseline wander removal process on the two sets of detected bioelectric potentials (Step S103). Then, the baseline-wander eliminator 12 eliminates extra high-frequency components from waveforms of the detected bioelectric potentials and performs a first derivation process on the waveforms after the high-frequency component is eliminated. Accordingly, the baseline-wander eliminator 12 eliminates the high-frequency components and baseline wanders.

Next, the electrocardiogram detector 13 detects the electrocardiogram with myoelectricity eliminated based on the outputs after the baseline wanders are removed (two sets of bioelectric potentials) (Step S104). Then, the electrocardiogram detector 13 subtracts the bioelectric potential not including electrocardiogram from the bioelectric potential including electrocardiogram among two sets of the bioelectric potentials. Thus, the electrocardiogram detector 13 detects an electrocardiogram with myoelectricity eliminated.

Next, the electrocardiograph 100 of the embodiment determines whether the electrocardiographic measurement of the examinee is terminated or not (Step S105).

As a result, if the electrocardiographic measurement is determined as not being terminated (Step S105: NO), the electrocardiograph 100 of the embodiment returns to the process of Step S102 and continues the electrocardiographic measurement process.

On the other hand, in the electrocardiograph 100 of the embodiment, when the electrocardiographic measurement is determined as terminated (Step S105: YES), the display controller 14 displays the measurement result, for example, the detected electrocardiographic complex, on the display device 106 (Step S106).

As described above, with the electrocardiograph 100 of the embodiment, at least two sets of the electrode pairs 1 and 2 of: the measurement electrode 1 and the reference electrode 1; and the measurement electrode 2 and the reference electrode 2 are disposed on the electrode-fitting surface considering the direction of the muscle fiber and the transmission direction of electrocardiogram at the installation position on the body member of the examinee. In the electrocardiograph 100 of the embodiment, the bioelectric potential detector 11 detects each differential electric potential between the electrodes of the measurement electrode 1 and the reference electrode 1 and between the electrodes of the measurement electrode 2 and the reference electrode 2 as two sets of bioelectric potentials. In the electrocardiograph 100 of the embodiment, the electrocardiogram detector 13 performs a subtraction process on the two sets of bioelectric potentials and detects an electrocardiogram.

Accordingly, the electrocardiograph 100 of the embodiment can prevent generation of myoelectricity by motion of the muscle fiber at the installation position and reduction in measurement accuracy due to smallness of detected bioelectric potentials compared with the electrocardiographic measurement sandwiching the heart with electrodes.

Thus, the electrocardiograph 100 according to the embodiment allows the examinee to perform electrocardiographic measurement in daily life without burden. The electrocardiograph 100 of the embodiment can improve measurement accuracy.

The above-described embodiment describes the configuration achieving the electrocardiographic measurement function by execution of the electrocardiographic program. However, this should not be construed in a limiting sense. The electrocardiographic measurement function may be achieved by various hardware, for example, functionality of the bioelectric potential detector 11 may be achieved with a differential amplifier circuit and functionality of the baseline-wander eliminator 12 may be achieved with a low-pass filter circuit.

The above-described embodiment describes an exemplary display method as a method for notifying the measurement result of electrocardiogram of the examinee. However, this should not be construed in a limiting sense. In the case where the electrocardiograph 100 of the embodiment includes a communication interface (IF: not illustrated), for example, the measurement result of the electrocardiogram may be transmitted to an external device via a network to notify the measurement result of the electrocardiogram. It should be understood that the “network” is irrespective of communications scheme, wired or wireless, etc. It is only necessary that an external terminal is a device with a communication function, such as a mobile phone or an information terminal.

The following describes a modification of the electrocardiograph 100 of the embodiment. Like reference numerals designate corresponding or identical elements throughout the following modification and the embodiment, and therefore such elements will not be further elaborated here.

Modification 1

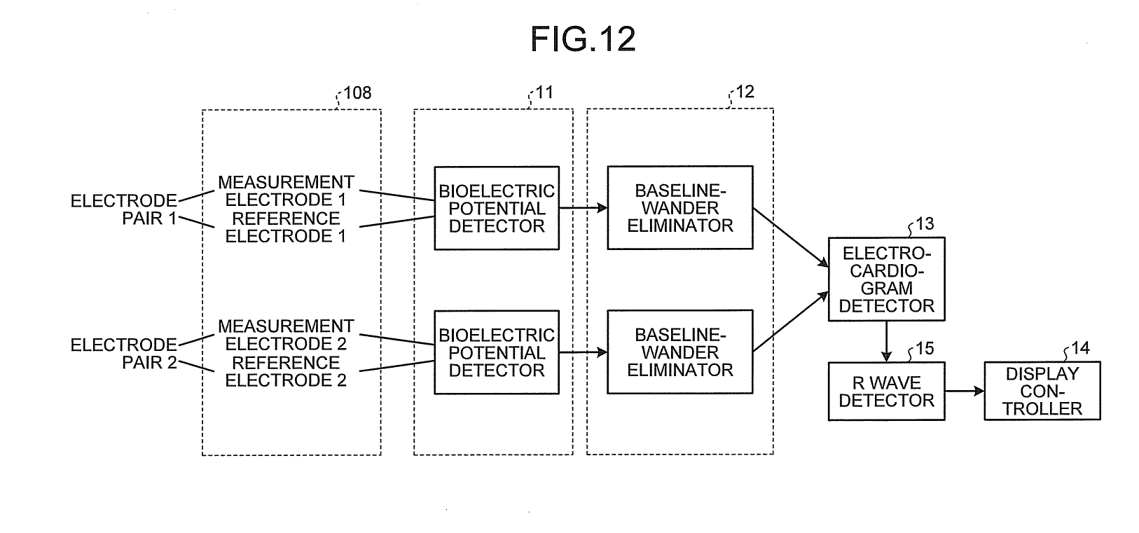

FIG. 12 illustrates an exemplary functional configuration of electrocardiographic measurement of Modification 1. As illustrated in FIG. 12, the electrocardiograph 100 of Modification 1 may include an R wave detector 15 to detect an R wave of the electrocardiogram.

The R wave detector 15 of Modification 1 detects an R wave from the electrocardiographic complex detected by the electrocardiogram detector 13. The R wave detector 15 detects the R wave by the following method. The R wave detector 15 sets, for example, 1.5 [sec] as detection time width and detects a local maximal value V of the electrocardiographic complex in the detection time. Then, the R wave detector 15 detects the R wave by the following Conditional expression.

V≧μ+α+σ

where V is a local maximal value of the electrocardiographic complex, μ is an average value of the local maximal value of the detected electrocardiographic complex, σ is a variance, α is a coefficient (for example, 0.8), and μ+α×σ represents a threshold.

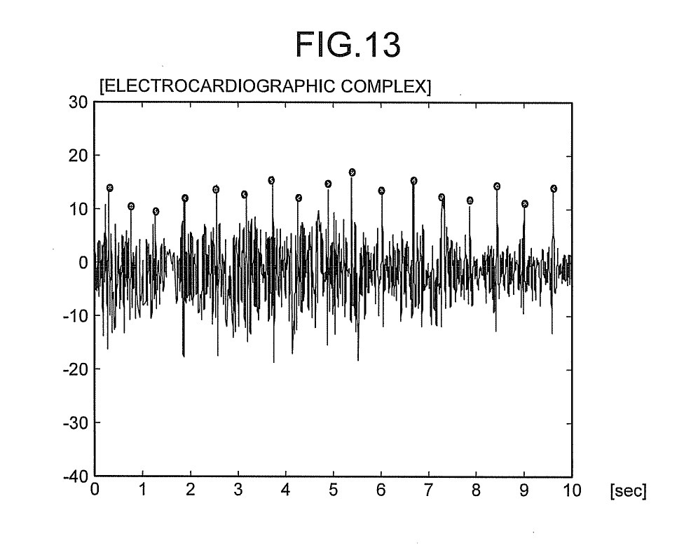

FIG. 13 illustrates an exemplary waveform of an electrocardiogram of Modification 1. As illustrated in FIG. 13, the R wave detector 15 of Modification 1 detects the local maximal values V equal to or more than the threshold (circular marks in the drawing) as R wave among the local maximal values V of the detected electrocardiographic complex. The method for detecting the R wave may not be the detection method with variable threshold using the detection time width but may be a method using fixed threshold, for example.

Thus, the electrocardiographic measurement function of Modification 1 outputs the electrocardiographic complex detected by the electrocardiogram detector 13 from the electrocardiogram detector 13 to the R wave detector 15. The R wave detector 15 detects the local maximal value V equal to or more than the threshold as illustrated in FIG. 13 as an R wave among the local maximal values V of the detected electrocardiographic complex. Afterwards, the detection result of the R wave is output from the R wave detector 15 to the display controller 14. As a result, the display controller 14 displays the detection result of the R wave on the display device 106. A method for notifying the detection result of the R wave of the examinee is not limited to the display. The method may be, for example, an alarm sound notifying detection of the R wave. The content of the result notified of the examinee may not be only the detection result of the R wave. The detection result of the electrocardiogram and the detection result of the R wave may be notified together, for example. The notification may be made only in case of failure in the detection result of the R wave, for example.

As described above, the electrocardiograph 100 of Modification 1 can notify not only the detection result of electrocardiogram but also the detection result of R wave of the examinee.

Modification 2

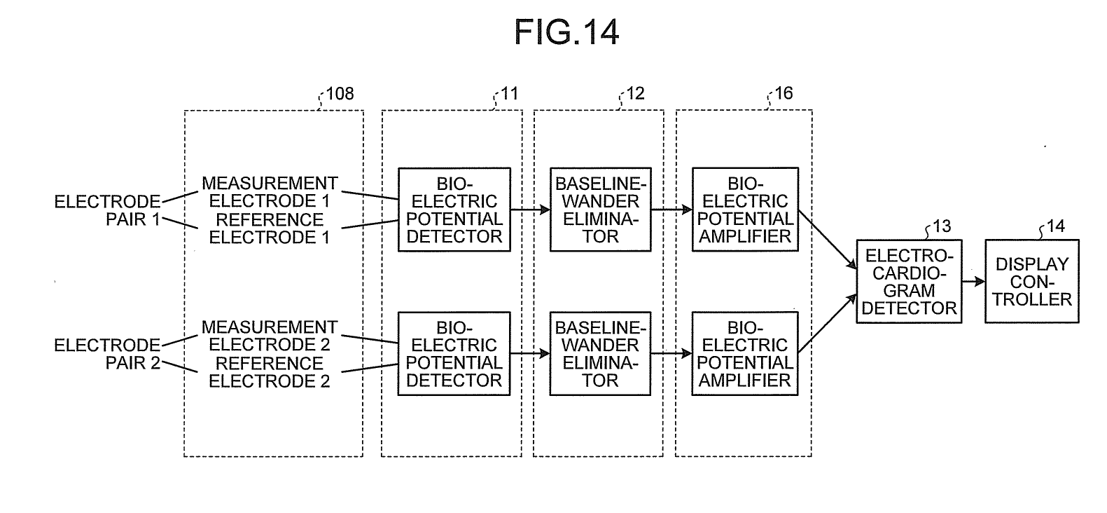

FIG. 14 illustrates an exemplary functional configuration of electrocardiographic measurement of Modification 2. As illustrated in FIG. 14, the electrocardiograph 100 of Modification 2 may include a bioelectric potential amplifier 16 that amplifies bioelectric potentials.

The bioelectric potential amplifier 16 of Modification 2 amplifies the bioelectric potential in accordance with the magnitude of amplitude of myoelectricity component included in the bioelectric potential after baseline wander is eliminated.

As described in the embodiment, the differential electric potential between the electrodes of the measurement electrode 1 and the reference electrode 1 and the differential electric potential between the electrodes of the measurement electrode 2 and the reference electrode 2 include the same level of myoelectricity. However, depending on installation state of the electrocardiograph 100, for example, the electrocardiograph 100 is installed at a position slightly shifted from the rectus abdominis muscle and the electrocardiograph 100 is inclinedly installed with respect to the vertical direction indicated with the mark M, amplitude of the myoelectricity included in each differential electric potential differs.

Therefore, in the electrocardiographic measurement function of Modification 2, the bioelectric potential amplifier 16 amplifies and corrects a bioelectric potential according to the magnitude of the amplitude of the myoelectricity component included in the bioelectric potential after baseline wander is eliminated.

The bioelectric potential amplifier 16 sets a period of 1.5 [sec] as detection time width for each two sets of output waveforms after baseline wander is eliminated, for example, and detects all the local maximal values and local minimal values of output waveforms detected in the detection time. The bioelectric potential amplifier 16 obtains an average value of differences between the adjacent local maximal values and local minimal values and sets the average value as a myoelectricity amplitude value. Accordingly, the bioelectric potential amplifier 16 obtains two sets of myoelectricity amplitude values corresponding to the output waveform measured from the electrode pair 1 of the measurement electrode 1 and the reference electrode 1 and the output waveform measured from the electrode pair 2 of the measurement electrode 2 and the reference electrode 2, respectively.

As a result, the bioelectric potential amplifier 16 amplifies the output waveform corresponding to the electrode pair 1 of the measurement electrode 1 and the reference electrode 1 according to Amplification factor 1 calculated by the following Equation (1).

Amplification factor 1=Myoelectricity amplitude value 2/(Myoelectricity amplitude value 1+Myoelectricity amplitude value 2) (1)

where Myoelectricity amplitude value 1 is a myoelectricity amplitude value corresponding to the electrode pair 1 of the measurement electrode 1 and the reference electrode 1, and Myoelectricity amplitude value 2 is a myoelectricity amplitude value corresponding to the electrode pair 2 of the measurement electrode 2 and the reference electrode 2.

The bioelectric potential amplifier 16 amplifies an output waveform corresponding to the electrode pair 2 of the measurement electrode 2 and the reference electrode 2 according to Amplification factor 2 calculated by the following Equation (2).

Amplification factor 2=Myoelectricity amplitude value 1/(Myoelectricity amplitude value 1+Myoelectricity amplitude value 2) (2)

The amplification factor should not be construed in a limiting sense. The amplification factor may be an amplification factor predetermined in accordance with the magnitude of myoelectricity amplitude, for example.

Thus, in the electrocardiographic measurement function of Modification 2, when the bioelectric potential amplifier 16 performs the amplifying process, two sets of bioelectric potentials after amplification is output from the bioelectric potential amplifier 16 to the electrocardiogram detector 13.

As described above, even if the amplitude of myoelectricity included in the two sets of differential electric potentials detected from each electrode pair 1 and 2 differs, the electrocardiograph 100 of Modification 2 prevents degrade of measurement accuracy by performing correction before electrocardiogram detection.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An electrocardiograph, comprising:
a first electrode pair including a first measurement electrode and a first reference electrode, the first measurement electrode being apart from the first reference electrode with a first distance on a first line;
a second electrode pair including a second measurement electrode and a second reference electrode, the second measurement electrode being apart from the second reference electrode with a second distance on a second line, a difference between the first distance and the second distance being equal to or less than a first threshold, an angle formed by the first line related to the first electrode pair and the second line related to the second electrode pair being equal to or more than a second threshold;
a first electric potential detector configured to detect a first differential electric potential of the first electrode pair;
a second electric potential detector configured to detect a second differential electric potential of the second electrode pair; and
an electrocardiogram detector configured to detect an electrocardiogram by performing a subtraction process on the first differential electric potential and the second differential electric potential.

2. The electrocardiograph according to claim 1, wherein
the first electrode pair is disposed vertical to a transmission direction of electrocardiogram while the second electrode pair is disposed horizontal to the transmission direction of electrocardiogram during installation of the electrocardiogram, and
the electrocardiogram detector performs the subtraction process that detects an electrocardiogram.

3. The electrocardiograph according to claim 1, wherein
each of the first distance and the second distance is equal to or smaller than a cross-sectional width of muscle fiber.

4. The electrocardiograph according to claim 1, wherein
an opposite surface of an electrode-fitting surface on which the first electrode pair and the second electrode pair are disposed has a mark thereon, the mark showing a vertical direction during installation of the electrocardiograph.

5. The electrocardiograph according to claim 1, further comprising an attachment configured to secure the electrocardiograph, the attachment being disposed on an opposite surface of an electrode-fitting surface on which the first electrode pair and the second electrode pair are disposed.

6. The electrocardiograph according to claim 1, further comprising:
a first electric potential amplifier configured to amplify the first differential electric potential based on an amplification factor according to a magnitude of an amplitude of myoelectricity component included in the first differential electric potential; and
a second electric potential amplifier configured to amplify the second differential electric potential based on an amplification factor according to a magnitude of an amplitude of myoelectricity component included in the second differential electric potential.

7. The electrocardiograph according to claim 1, wherein
the first electric potential detector is configured to obtain a difference between an electric potential measured at the first measurement electrode and an electric potential measured at the first reference electrode to detect the first differential electric potential, and
the second electric potential detector is configured to obtain a difference between an electric potential measured at the second measurement electrode and an electric potential measured at the second reference electrode to detect the second differential electric potential.

8. A method for measuring electrocardiogram by an electrocardiograph that includes a first electrode pair and a second electrode pair, the first electrode pair including a first measurement electrode and a first reference electrode, the first measurement electrode being apart from the first reference electrode with a first distance on a first line, the second electrode pair including a second measurement electrode and a second reference electrode, the second measurement electrode being apart from the second electrode with a second distance on a second line, a difference between the first distance and the second distance being equal to or less than a first threshold, an angle formed by the first line related to the first electrode pair and the second line related to the second electrode pair being equal to or more than a second threshold, the method comprising:
detecting a first electric differential electric potential of the first electrode pair;
detecting a second electric differential electric potential of the second electrode pair; and
detecting an electrocardiogram by performing a subtraction process on the first differential electric potential and the second differential electric potential.

9. A computer program product comprising a computer-readable medium containing a program executed by a computer, the program causing the computer to execute:
detecting a first electric differential electric potential of a first electrode pair, the first electrode pair including a first measurement electrode and a first reference electrode, the first measurement electrode being apart from the first reference electrode with a first distance on a first line;
detecting a second electric differential electric potential of a second electrode pair, the second electrode pair including a second measurement electrode and a second reference electrode, the second measurement electrode being apart from the second reference electrode with a second distance on a second line, a difference between the first distance and the second distance being equal to or less than a first threshold, an angle formed by the first line related to the first electrode pair and the second line related to the second electrode pair being equal to or more than a second threshold; and
detecting an electrocardiogram by performing a subtraction process on the first differential electric potential and the second differential electric potential.
